(19) United States
(12) Patent Application Publication
(10) Pub. No.: US 2022/0057370 A1
(43) Pub. Date: Feb. 24, 2022

(54) FILTER FOR CHEMICAL REACTORS

(71) Applicant: PHARMAFLUIDICS NV, Gent (BE)

(72) Inventors: Jeff OP DE BEECK, Mariakerke (BE); Bo CLAEREBOUT, Kortrijk (BE); Paul JACOBS, Lokeren (BE)

(21) Appl. No.: 17/310,264

(22) PCT Filed: Jan. 31, 2020
(86) PCT No.: PCT/IB2020/050796

§ 371 Date: Jul. 27, 2021

(30) Foreign Application Priority Data
Jan. 31, 2019 (BE) 2019/5061

(51) Int. Cl. G01N 30/60; B01J 8/00; B01D 15/22

(52) U.S. Cl. CPC G01N 30/6017; B01J 8/006; G01N 2030/027; B01D 15/22; G01N 30/606; B01J 8/0085

(57) ABSTRACT

A chemical reactor is implemented on a substrate and has an inlet for receiving a fluid and/or a gas; a filter element for reducing or preventing that materials cause a blockage in the fluid supplied and/or the gas supplied in a part of the chemical reactor located further away; and a part located further away for transporting and/or processing the fluid and/or the gas. The part located further away has a depth dlow smaller than the depth dhigh of the inlet. The filter element has a first duct part and a second duct part; the first duct part is positioned closer up against the inlet than the second duct part, the first duct part is deeper than the second duct part, the first duct part has a diverging width and is free from pillar structures, and the second duct part is filled with filter pillars.

### SCOPE OF THE INVENTION

This invention generally relates to chemical reactors such as chromatographic systems for example. More specifically, the present invention relates to an inlet for chemical reactors, for example an inlet at a duct having pillar structures.

### BACKGROUND OF THE INVENTION

Systems that make use of liquid propagation have a large number of applications, including production of chemical components, synthesis of nanoparticles, separation and/or extraction of components, etc. A specific example of a separation technique for separating mixtures, for example for being able to accurately analyse them, is chromatography. There is a variation in forms of chromatography such as gas chromatography, gel chromatography, thin-coating chromatography, adsorption chromatography, affinity chromatography, liquid chromatography, etc. Liquid chromatography is typically used in pharmacy and chemistry, for both analytical and production applications. In liquid chromatography, use is made of the difference in solubility of various substances having a mobile phase and a stationary phase. As each substance has its own “bonding power” to the stationary phase, they are moved along faster or slower with the mobile phase and as such, certain substances may be separated from other ones. In principle, it is applicable to any connection, having the advantage that no evaporation of the material is required and that variations in temperature only have a negligible effect.

A typical example of liquid chromatography is based on chromatographic columns on the basis of multiple ducts interconnected in series in which the separation of the phases may be achieved for practical applications.

It is well known that various problems may manifest at the inlet at these ducts.

One of the known problems is accurately mounting the various components in the chromatographic column, like for example mounting the capillary which supplies the fluid in the duct, relating to the inlet duct in the chemical reactor which is implemented on a substrate.

A second known problem relates to partly or partially blocking the inlet at the entrance of the duct. This phenomenon often occurs at the level of the distributor which has as its function the widening of the fluid plug, to the width of the duct in which the separation occurs.

### SUMMARY OF THE INVENTION

It is an objective of embodiments according to the present invention to produce good systems for separating materials.

It is an advantage of some embodiments of the present invention that one or several problems of systems according to the state of the art are resolved.

The preceding objective may be achieved by a device according to embodiments of the present invention.

In a first aspect, the present invention relates to a chemical reactor implemented on a substrate, the chemical reactor comprising

an inlet for receiving a fluid and/or a gas, whereby the inlet has a first depth d_{high} and has been adjusted to accommodate a capillary,
a filter element for reducing or preventing that materials cause a blockage in the fluid and/or gas supplied in a part of the chemical reactor located further away, and
a part located further away for transporting and/or processing the fluid and/or the gas, whereby the part located further away has a depth d_{low} smaller than depth d_{high} of the inlet,
characterised in that
the filter element comprises a first duct part and a second duct part, whereby the first duct part is positioned closer up against the inlet than the second duct part, the first duct part is deeper than the second duct part, or in other words, the first duct part has a depth that is greater than a depth of the second duct part, and the first duct part has a diverging width and is free from pillar structures, and the second duct part is filled with filter pillars. Hereby, the diverging width of the first duct part is a widening of the width of the first duct part in downstream direction, i.e. from the duct inlet towards the part located further away.

In a related aspect, the present invention also relates to a design for a chemical reactor as described above.

Specific and preferable aspects of the invention have been included in the attached independent and dependent claims. Features of the dependent claims may be combined with features of the independent claims and with features of other dependent claims such as indicated and not only as expressly brought forward in the claims.

In a second aspect, the present invention relates to a chemical reactor implemented on a substrate, the chemical reactor comprising

an inlet duct adjusted to accommodate a capillary for supplying fluid and/or gas to a separation duct,
a distributor to check the transition in width of the fluid and/or gas plug between the capillary and the separation duct, and
a separation duct, which optionally comprises pillar structures,
whereby the inlet duct is provided with a stop element for accurately positioning the capillary in the inlet duct.

In a related aspect, the present invention also relates to a design for a chemical reactor as described above.

In another aspect, the present invention also relates to a chemical reactor, in which, in a duct leading to a part of the chemical reactor located further away, a higher density of pillar structures is provided locally. In a related aspect, the present invention also relates to a design for a chemical reactor as described above.

### SHORT DESCRIPTION OF THE FIGURES

FIG. 1 illustrates a first design for a chemical reactor according to an embodiment of the present invention.

FIG. 2 illustrates the effect of the chemical reactor according to FIG. 1 on the blockage in the system.

FIG. 3 illustrates a second design for a chemical reactor according to embodiments of the present invention.

FIG. 4 illustrates the effect of the chemical reactor according to FIG. 1 on blockage in the system.

FIG. 5 illustrates a chemical reactor locally having higher density of pillar structures in a duct, according to an embodiment of the present invention.

The figures are only schematic and not restrictive. It is possible that, for illustrative purposes, the dimensions of some components are exaggerated and not represented to scale in the figures. The dimensions and relative dimensions do not necessarily correspond with the ones from practical embodiments of the invention. Reference numbers used in the claims may not be interpreted to restrict the scope of protection.

### DETAILED DESCRIPTION OF ILLUSTRATIVE EMBODIMENTS

The present invention will be described referring to specific embodiments and to certain figures, but the invention is not restricted by them and is only restricted by the claims.

It should be noted that the terms “contain” and “comprise”, as used in the claims, should not be interpreted as being restricted to the items described thereafter; these terms do not exclude any other elements or steps. Therefore, they may be interpreted as specifying the presence of the features, values, steps or components indicated which are referred to, but do not exclude the presence or addition of one or several other features, values, steps or components, or groups thereof. So, the extent of the expression “a device containing items A and B” should not be restricted to devices consisting of components A and B only. It means that in respect of the present invention, A and B are the only relevant components of the device.

References throughout this specification to “one embodiment” or “an embodiment” mean that a specific feature, structure or characteristic described in connection with the embodiment has been included in at least one embodiment of the present invention. Therefore, occurrences of the expressions “in one embodiment” or “in an embodiment” in various locations throughout this specification do not necessarily all need to refer to the same embodiment but may do so. Furthermore, the specific features, structures or characteristics may be combined in any suitable manner, as would be clear to a person skilled in the art on the basis of this publication, in one or several embodiments.

Similarly, it should be appreciated that in the description of sample embodiments of the invention, various features of the invention are sometimes grouped together in one single embodiment, figure or description thereof intended to streamline the publication and to help the understanding of one or several of the various inventive aspects. This method of publication should therefore not be interpreted as a reflection of an intention that the invention requires more features than explicitly mentioned in each claim. Rather, as the following claims reflect, inventive aspects lie in fewer than all features of one single previously publicised embodiment. Therefore, the claims following on from the detailed description have been explicitly included in this detailed description, with every independent claim being a separate embodiment of this invention.

Furthermore, while some embodiments described herein contain some, but not other, features included in other embodiments, combinations of features from various embodiments are intended to be within the scope of the invention, and they form various embodiments as would be understood by the person skilled in the art. For example, in the following claims, any of the embodiments described may be used in any combination.

When, in embodiments of the present invention, reference is made to depth, reference is made to the dimension measured perpendicular to the substrate on which the chemical reactor is implemented.

When, in embodiments of the present invention, reference is made to a part located further away, reference is made to a part that is downstream in the chemical reactor. This may be a trapping column for example, but also another micro-fluidic element.

When, in embodiments of the present invention, reference is made to a diverging width of the first duct part, reference is made to all possible duct parts the width of which is smaller at the entrance than the width at the exit. In some embodiments, the width in the direction of the direction of flow may systematically increase, in some embodiments, the width in a piece of the duct part may systematically increase but also non-monotonous or strictly monotonous widenings of the duct may occur and come under the term diverging width.

In a first aspect, the present invention relates to a chemical reactor. Such a chemical reactor may be a chromatographic column but is not restricted to this. Other examples of chemical reactors which may derive advantage from the present inventions may be enrichment filters or trapping columns for example, reactors with (micro) catalysts, multi-phase reactors, fuel cells, electrochemical reactors, reactors for capillary electrochromatography, etc. The present invention relates to a chemical reactor implemented on a substrate.

The chemical reactor comprises an inlet for receiving a fluid and/or gas. Such an inlet is typically a micro-fluidic duct, in which a capillary is introduced along which the fluid and/or the gas are/is supplied in the chemical reactor. The inlet duct typically has a depth d_{high}.

According to embodiments of the present invention, the chemical reactor also has a filter element for reducing or preventing that materials cause a blockage in the fluid supplied and/or the gas supplied in a part of the chemical reactor located further away. As blockages are one of the main causes for chemical reactors not to function accurately, this filter element results in important advantages relating to efficiency of these systems as well as accuracy of these systems.

In addition to the filter element, there is also at least one part located further away (a part located further downstream compared with the inlet and the filter element) which may be used for transporting and/or treating the fluid and/or the gas for example, for separating different phases from the fluid and/or the gas for example. This part located further away typically has a depth d_{low} smaller than depth d_{high} of the inlet.

Embodiments according to this first aspect of the present invention are further characterised by the fact that the filter element comprises a first duct part and a second duct part. The first duct part is positioned closer up against the inlet than the second duct part. Furthermore, the first duct part is also deeper than the second duct part. The first duct part also has a diverging width and is free from pillar structures. The second duct part is filled with filter pillars.

In some embodiments, the filter element shows a sudden jump, also described as a step, in depth so that the filter element induces a filtering effect. It is an advantage of embodiments of the present invention that a filter element the in which depth shows a sudden jump surprisingly induces a filtering effect to this jump. As a result, the chance that waste from during the production of the chemical reactor or other interfering elements cause a blockage in the chemical reactor is smaller because this waste or these interfering elements do not reach the fine passages in the reactor parts located further away (as they are held back earlier, in the sudden jump in depth for example).

In some embodiments, the chemical reactor is adjusted, whereby the inlet and the filter element have been constructed such that, when the capillary is positioned in the inlet, the fluid and/or the gas supplied by the capillary has a drop in the first duct part of the filter element. The capillary is normally glued into the inlet, so that the capillary is positioned in the chemical reactor each time a system is functioning. It is an advantage of embodiments of the present invention that the depth of the first duct part may be selected in function of the thickness of the capillary wall used, so that additional turbulence is created in the first duct part.

The depth of the inlet and/or of the first duct part may be, for example, between 80 μm and 200 μm, for example, between 100 μm and 150 μm. The depth of the second duct part may, for example, be between 10 μm and 60 μm, for example, between 15 μm and 40 μm. This may match the depth of the part located further away. The transition between the various depths may be sudden, i.e. by means of one or several steps. In some embodiments, the transition may also be provided gradually.

In some embodiments, the depth of the first duct part is equal to depth d_{high} of the inlet and/or the depth of the second duct part is equal to the depth d_{low} of the part located further away. It is an advantage of embodiments of the present invention that the number of different depths, which must be generated in the capillary, may be restricted. When these are produced by etching for example, it is an advantage that the duct parts of the filter element may have the same depth as the inlet and the part located further away.

In some embodiments, the filter pillars have a length/width aspect ratio between 2 and 0.5, for example, between 1.2 and 0.8. Where, in embodiments of the present invention, reference is made to a length/width aspect ratio, reference is made to the dimension of the pillars in the longitudinal direction of the duct, i.e. in the average direction of the fluid or gas flow compared with the dimension in the width direction of the duct, i.e. in the direction perpendicular to the side walls.

In some embodiments, the filter pillars are cylindrical. It is an advantage of embodiments of the present invention that the use of cylindrical filter pillars allows for a large number of intermediate ducts to be generated in the filter element, while the space needed for filtering may be limited in favour of the length of a separation bed for example, which follows after the filter element.

In some embodiments, there are also pillar structures present in the part located further away. The smallest distance between the filter pillars and the second duct part is, at most, the distance between the pillar structures in the part located further away. It is an advantage of embodiments of the present invention that the specific distance between pillars in the filter element may result in the fact that blockages do not occur in parts located further away in the reactor, which are also based on pillar structures.

In some embodiments, the number of filter pillars in the first row transversely to the duct which is reached downstream from the inlet is at least 5, for example, at least 7, for example, at least 9, for example, at least 11, for example, at least 13, for example, at least 15. It is an advantage of embodiments of the present invention that the number of ducts through which the fluid and/or gas may flow is initially large, so that blockage of one or several ducts does not lead to immediate blockage of the entire reactor.

In some embodiments, the second duct part comprises a first set of cylindrical filter pillars positioned closer up against the inlet and comprises a second set of cylindrical filter pillars positioned further away from the inlet compared with the first set, whereby the first set contains larger filter pillars, having a larger diameter than the diameter of the filter pillars in the second set. In embodiments of the present invention, more than two sets of filter pillars with different diameters may be used too.

In some embodiments, the part located further away is a separation duct.

In some embodiments, the separation duct is filled with elongated pillars orientated such that the longitudinal direction is perpendicular to the average direction of flow in the separation duct or in which the separation duct is filled with cylindrical pillars.

In some embodiments, the inlet is provided with a stop element for accurately positioning the capillary in the inlet duct. It is an advantage of embodiments of the present invention that the mounting of the capillary in the chemical reactor may occur in a controlled manner, so that the risk of damage is restricted. As a stop element is provided in the inlet duct, the capillary cannot cause damage to parts of the distributor or of the separation duct when installing the capillary.

It is an advantage of embodiments of the present invention that the mounting of the capillary in the chemical reactor may occur in an efficient manner.

In some embodiments, the stop element is formed by a narrowing of the inlet duct.

The chemical reactor may comprise a chromatographic column. The chemical reactor may be a chromatography system. The chromatography system may be a high-performance fluid chromatography system.

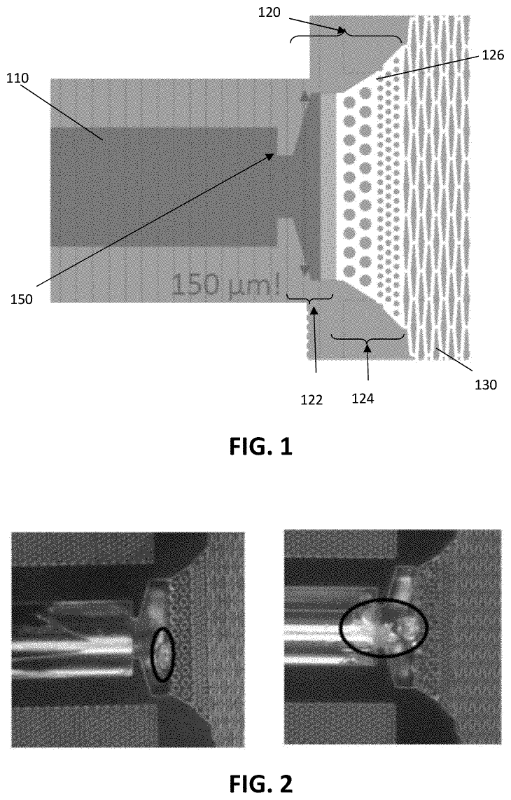

By way of illustration, two examples are shown of chemical reactors having a filter element according to the present invention, referring to FIG. 1 tot FIG. 4, although embodiments are of course not restricted by this.

FIG. 1 illustrates a chemical reactor showing the inlet 110, the filter element 120 having a first duct part 122 and a second duct part 124. In the second duct part 124, which is the part of the filter element 120 that has the smallest depth, filter pillar structures 126 are provided. In the current example, the part 130 located further away is a trapping column. In the present example, the trapping column itself is also provided with pillars, also referred to as pillar structures, whereby the ones in the present example have an elongated form orientated transversely to the direction of flow. However, it should be noted that the present invention is not restricted by this and may be applied for elements located further away with other pillar structures.

In the present example, the filter element is also a distributor which ensures that the fluid and/or gas plug to be treated or analysed widens the width determined by the capillary in which it is supplied and the width of the trapping column itself. In the present example, the part 122 has the same depth as the inlet, while the part 124 has the same depth as the trapping column. In the present example, the depth of the first duct part is approximately 130 μm and the depth of the second duct part is approximately 20 μm. As a result, the filter element 120 provides a transition in depth, so that a filter function is generated. In FIG. 1 a stop part is provided too for accurately installing the capillary in the chemical reactor. The capillary which is typically just a little smaller than the diameter of the inlet may then be slid into the inlet and is spontaneously blocked when the stop part 150 is reached.

FIG. 2 illustrates for a system that is schematically shown in FIG. 1, that the sticking together of the material typically occurs in the first duct part and the materials or debris causing this therefore do not end up in the column itself, so that they cannot cause any blockage. The delineated parts in the photo show the stuck-together material.

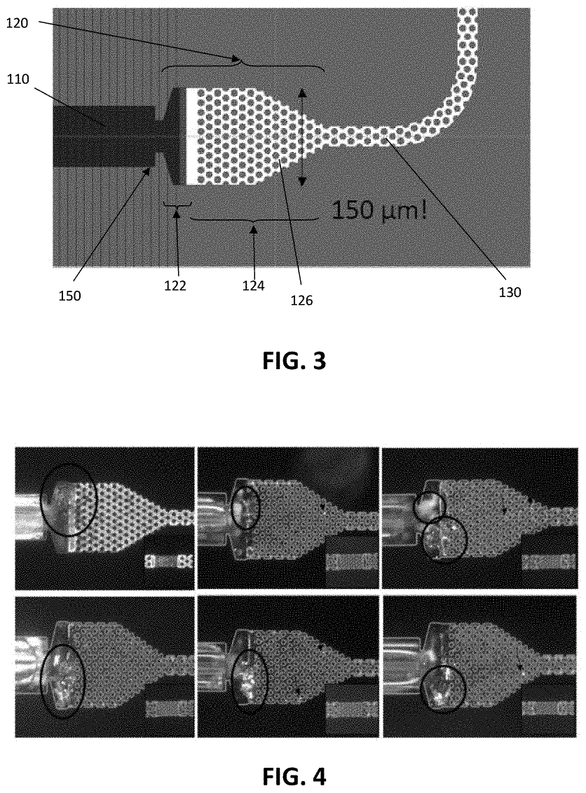

In FIG. 3, an alternative example of a chemical reactor is shown. In this example, the part located further away is not a trapping column but a duct provided with pillar structures. However, the filter function provided by filter element 120 functions in the same manner.

FIG. 4 illustrates for a system that is schematically shown in FIG. 3 that the sticking together of the material typically occurs in the first duct part and the materials or debris, causing this, therefore do not end up in the duct itself, so that they cannot cause any blockage.

In a related aspect, the present invention relates to a design for a chemical reactor as described in the aspect above.

In another aspect, the present invention relates to a chemical reactor implemented on a substrate, the chemical reactor comprising

an inlet duct adjusted to accommodate a capillary for supplying fluid and/or gas to a separation duct,
a distributor to check the transition in width of the fluid and/or gas plug between the capillary and the separation duct, and
a separation duct, which optionally comprises pillar structures,
whereby the inlet duct is provided with a stop element for accurately positioning the capillary in the inlet duct. This is an action which typically occurs once at installation.

It is an advantage of embodiments of the present invention that the mounting of the capillary in the chemical reactor may occur in a controlled manner, so that the risk of damage is restricted. As a stop element is provided in the inlet duct, the capillary cannot cause damage to parts of the distributor or of the separation duct.

It is an advantage of embodiments of the present invention that the mounting of the capillary in the chemical reactor may occur in an efficient manner.

The stop element may be formed by a narrowing of the inlet duct. This may be formed by locally giving the inlet duct a different etching depth. The stop material may be constructed from the same material as the material from which the inlet duct is made, although this is not essential.

The inlet duct may be substantially deeper than the separation duct.

It is an advantage of embodiments of the present invention that the inlet duct may easily accommodate the capillary.

Although stop elements are illustrated for the chemical reactors presented in FIG. 1 and FIG. 3, chemical reactors according to this aspect of the present invention must not comprise any filter element as described in the earlier aspects. The stop element may be provided separately from this.

In a related aspect, the present invention also relates to a design for a chemical reactor as described above.

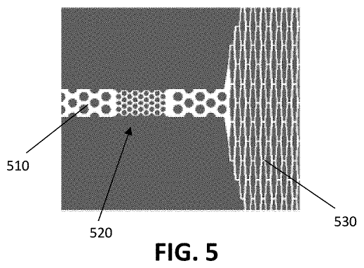

In yet another aspect, the present invention also relates to a chemical reactor, in which, in a duct leading to a part of the chemical reactor located further away, a higher density of pillar structures is provided locally. This may happen, for example, by selecting a smaller average diameter of the pillar structures in this part. The pillar structures are preferably arranged such that a larger number of passageways for the fluid or the gas are provided locally. As an illustration, this structure is shown in FIG. 5 showing a duct 510, a part 520 in the duct 510 having a higher density of pillar structures, and a part 530 located further away. The density may, for example, be twice as high, three times as high, etc. The advantages of the use of a part having higher density of pillar structures are the fact that additional mixing occurs (due to the higher number of confluence points) of the fluid and/or gas plug so that, even if a blockage occurs in one of the passages of the first row of pillars from the part 520, the spreading of the fluid or gas plug in the part 530 located further away, e.g. the distributor, will occur in a uniform manner due to it being able to retain its full functionality. It should be noted that cylindrical pillar structures are often used, but that the present invention is not limited by this and other forms may be used too. In a related aspect, the present invention also relates to a design for a chemical reactor according to the aspect above.

1.-16. (canceled)

17. A chemical reactor implemented on a substrate, the chemical reactor comprising:
an inlet for receiving a fluid and/or a gas, wherein the inlet has a first depth dhigh and has been adjusted to accommodate a capillary,
a filter element for reducing or preventing that materials cause a blockage in the fluid and/or gas supplied in a part of the chemical reactor located further away, and
a part located further away for transporting and/or processing the fluid and/or the gas, wherein the part located further away has a depth dlow smaller than depth dhigh of the inlet,
wherein
the filter element comprises a first duct part and a second duct part, wherein
the first duct part is positioned closer up against the inlet than the second duct part,
the first duct part has a greater depth than a depth of the second duct part,
the first duct part has a diverging width so that the first duct part has a widening of the width in a downstream direction from the duct inlet towards the part located further away and the first duct part is free from pillar structures, and
the second duct part is filled with filter pillars.

18. The chemical reactor according to claim 17, in which the filter element shows a sudden step in depth inducing a filtering effect.

19. The chemical reactor according to claim 17, in which the inlet and the filter element are constructed such that, when the capillary is positioned in the inlet, the fluid and/or the gas supplied by the capillary has a drop in the first duct part of the filter element.

20. The chemical reactor according to claim 17, in which the depth of the first duct part is equal to depth dhigh of the inlet and/or in which the depth of the second duct part is equal to the depth dlow of the part located further away.

21. The chemical reactor according to claim 17, in which the filter pillars have a length/width aspect ratio between 2 and 0.5, for example between 1.2 and 0.8.

22. The chemical reactor according to claim 17, in which the filter pillars are substantially cylindrical.

23. The chemical reactor according to claim 17, in which pillar structures are present too in the part located further away and wherein the smallest distance between the filter pillars in the second duct part is at most the distance between the pillar structures in the part located further away.

24. The chemical reactor according to claim 17, wherein the number of filter pillars in the first row transversely to the duct which is reached downstream from the inlet is at least 5, for example, at least 7, for example, at least 9, for example, at least 11, for example, at least 13, for example, at least 15.

25. The chemical reactor according to claim 17, in which the second duct part comprises a first set of cylindrical filter pillars positioned closer up against the inlet and comprises a second set of cylindrical filter pillars positioned further away from the inlet compared with the first set,
wherein the first set contains larger filter pillars having a larger diameter than the diameter of the filter pillars in the second set.

26. The chemical reactor according to claim 17, in which the part located further away may be a separation duct.

27. The chemical reactor according to claim 17, in which the separation duct is filled with elongated pillars orientated such that the longitudinal direction is perpendicular to the average direction of flow in the separation duct or in which the separation duct is filled with cylindrical pillars.

28. The chemical reactor according to claim 17, in which the inlet is provided with a stop element for accurately positioning the capillary in the inlet duct.

29. The chemical reactor according to claim 28, in which the stop element is formed by a narrowing of the inlet duct.

30. The chemical reactor according to claim 17, in which the chemical reactor comprises a chromatographic column.

31. The chemical reactor according to claim 17, in which the chemical reactor is a chromatography system.

32. The chemical reactor according to claim 31, in which the chromatography system is a high-performance fluid chromatography system.
